(19) United States
(12) Patent Application Publication
(10) Pub. No.: US 2021/0059962 A1
(43) Pub. Date: Mar. 4, 2021

(54) STIMULATION OF T REGULATORY CELLS BY CANNABIDIOL AS A MEANS OF TREATING ARTHRITIS AND AUTOIMMUNITY

(71) Applicant: Regen Biopharma, Inc., La Mesa, CA (US)

(72) Inventors: Thomas Ichim, San Diego, CA (US); David Koos, La Mesa, CA (US)

(73) Assignee: Regen Biopharma, Inc., La Mesa, CA (US)

(21) Appl. No.: 17/010,720

(22) Filed: Sep. 2, 2020

Related U.S. Application Data
(60) Provisional application No. 62/895,340, filed on Sep. 3, 2019.

(51) Int. Cl. A61K 31/05; C07K 16/28; A61K 38/20; A61K 35/15; A61K 35/28; A61P 19/02

(52) U.S. Cl. CPC A61K 31/05; C07K 16/289; A61P 19/02; A61K 35/15; A61K 35/28; A61K 38/2013

(57) ABSTRACT

Disclosed are means, methods, and compositions of matter for stimulation of T regulatory (Treg) cells by cannabidiol. In one embodiment, administration of cannabidiol is performed in an animal suffering from autoimmunity. In situations of autoimmunity, cannabidiol induces generation of Treg cells while other agents or therapies may be used to expand such cells in vitro and/or in vivo. In some embodiments the administration of cannabidiol is performed together with agents such as low dose interleukin-2 in order to treat inflammatory conditions such as arthritis or other conditions associated with reduction in Treg cells such as pregnancy failure, type 1 diabetes, or graft versus host disease.

### CROSS-REFERENCE TO RELATED APPLICATIONS

This application claims the benefit of priority to U.S. Provisional Application No. 62/895,340, filed Sep. 3, 2019, the contents of which are incorporated herein by reference.

### BACKGROUND OF THE INVENTION

The treatment of autoimmune diseases is typically with immunosuppression to decrease the immune response. Examples include corticosteroids such as prednisone. Corticosteroids are known to cause osteoporosis and other side effects with extended use. Alternatively or additionally, non-steroid drugs such as azathioprine, cyclophosphamide, mycophenolate, sirolimus, tacrolimus, methotrexate, antibodies such as antibodies to tumor necrosis factor-alpha (TNF-.alpha.) or antibodies to certain cytokines may be used. Generally most of the current treatments are not effective in all cases and are known to have side effects with extended use. Despite the successful introduction of biologics such as TNF-.alpha. antibodies and beta interferon, therapy of autoimmune disease such as rheumatoid arthritis, juvenile idiopathic arthritis and multiple sclerosis is still ineffective in a sizable proportion of patients, who are exposed to a treatment they are bound to fail, with high social and economic costs. The treatment also lacks specificity, equally targeting detrimental autoimmune as well as beneficial responses, with obvious health hazards [Scott et al. (2010) Lancet 376: 1094-1108].

The utilization of T regulatory cells to suppress the immune system is much more enticing due to ability to antigen specifically suppress the immune system. Unfortunately, current means of expanding T regulatory cell in vivo are non-existent or not practical.

Immunological tolerance is a critical feature of the immune system, which allows for recognition and elimination of pathological threats, while selectively ignoring antigens that belong to the body. Understanding mechanisms of immunological tolerance, and having the ability to induce this process would make a major impact in autoimmune conditions, which affect approximately 8% of the US population.

Major autoimmune diseases include rheumatoid arthritis, multiple sclerosis, type 1 diabetes, systemic lupus erythromatosis, and inflammatory bowel disease. Traditionally, autoimmune conditions are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. Regardless, sales of TNF-alpha inhibitors have been quite successful: Humira ($9.2B; 2012), Enbrel ($7.8B; 2011), Remicade ($6.7B; 2011). These approaches do not “cure” autoimmunity, but merely alleviate symptomology.

To “cure” autoimmunity, it is essential to delete/inactivate the T cell clone that is recognizing the autoantigen in a selective manner. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

The invention seeks to replicate natural processes of tolerogenesis by administration of fibroblasts that are transfected with an autoantigen. For example it is known that tolerogenesis occurs in adults in settings such as pregnancy, cancer, and oral tolerance. The invention teaches utilization of molecules, cells and processes that occur in these situations in order to modify fibroblasts as a tolerogenic mediator.

### SUMMARY OF THE INVENTION

The teachings herein are directed to methods of inducing T regulatory cells in vivo comprising the steps of: a) providing cannabidiol to a mammal at a concentration sufficient to induce expression of Foxp3 in T cells possessing the marker CD4; and b) altering the dose of cannabidiol as needed in order to maintain an general increase in cells expressing CD4 and FoxP3. Treg cells can be further expanded in vivo by administration of low dose interleukin-2, by administration of antibody to CD45RB, and administration of immature dendritic cells.

Preferably the immature dendritic cells expression low levels CD40 as compared to steady state dendritic cells. Further embodiments include methods wherein immature dendritic cells expression low levels CD80 as compared to steady state dendritic cells. Further embodiments include methods wherein immature dendritic cells expression low levels CD86 as compared to steady state dendritic cells. Further embodiments include methods wherein immature dendritic cells expression low levels IL-12 as compared to steady state dendritic cells. Further embodiments include methods wherein immature dendritic cells expression higher levels of IL-10 as compared to steady state dendritic cells. Further embodiments include methods wherein immature dendritic cells expression higher levels of PD-L1 as compared to steady state dendritic cells. Further embodiments include methods wherein mesenchymal stem cells are administered together with cannabidiol. Further embodiments include methods wherein said mesenchymal stem cells are obtained from adipose tissue. Further embodiments include methods wherein said mesenchymal stem cells are obtained from bone marrow tissue. Further embodiments include methods wherein said mesenchymal stem cells are obtained from endometrial tissue. Further embodiments include methods wherein said mesenchymal stem cells are obtained from peripheral blood. Further embodiments include methods wherein said mobilization of mesenchymal stem cells is accomplished by administration of an agent selected from a group comprising of: a) G-CSF; b) GM-CSF; c) M-CSF; d) Mozibil; and e) FLT-3 ligand. Further embodiments include methods wherein said activation of Treg cells is accomplished in a mammal suffering from an inflammatory condition. Further embodiments include methods wherein said inflammatory condition is osteoarthritis. Further embodiments include methods wherein said inflammatory condition is rheumatoid arthritis. Further embodiments include methods wherein said inflammatory condition is an autoimmune disease.

### BRIEF DESCRIPTION OF THE DRAWINGS

FIG. 1 is a bar graph showing cannabidiol and IL-2 reverse rheumatoid arthritis.

FIG. 2 depicts stains evidencing that cannabidiol and IL-2 reverse rheumatoid arthritis.

FIG. 3 is a line graph showing cannabidiol and IL-2 increase production of T regulatory cells.

### DESCRIPTION OF THE INVENTION

The invention provides means of increasing T regulatory cell activity in a mammal through administration of cannabidiol alone, and/or together with adjuvants which enhance T regulatory cell generation, and/or proliferation, and/or activity. It is known that T regulatory cells play a role in the prevention and/or suppression of autoimmunity. The role of Treg cells is universally associated with tolerance in conditions of natural tolerance such as in pregnancy [4, 5], transplantation tolerance [6-10], and ocular tolerance [11-20]. The functional relevance of Treg cells to preservation and/or initiation of tolerance is observed in conditions where administration of Tregs prevents pathology, such as in spontaneous abortion [21].

In one embodiment of the invention, cannabidiol is administered together with low dose interleukin-2 for augmentation of T regulatory cell number and/or activity, in order to treat conditions associated with inflammation and/or autoimmunity. In some embodiments, administration of cannabidiol may be performed systemically and/or locally. In one embodiment of the invention, cannabidiol (CBD) is utilized to activate T regulatory cells in vitro prior to implantation. In other embodiments CBD is administered systemically with the two goals of: a) increasing suitability of the microenvironment for sustaining T regulatory cell activity; b) suppressing inflammation; and c) directly stimulating T regulatory cells to perform healing functions.

The CBD can be administered transdermally on the subject's upper arm and shoulder. In some embodiments, the CBD is administered transdermally on the subject's thigh or back. The CBD can be synthetic CBD. The CBD can be purified CBD. The CBD can be botanically derived. Transdermally administering an effective amount of cannabidiol (CBD) can reduce an intensity of at least one adverse event or side effect relative to orally administering CBD. The at least one adverse event or side effect can be a gastrointestinal (GI) adverse event. The at least one adverse event or side effect can be liver function. In some embodiments, the at least one adverse event is somnolence. In some embodiments, the frequency and intensity of somnolence is reduced as an adverse event.

In some embodiments, the CBD is (−)-CBD. The effective amount of CBD can be between about 50 mg to about 500 mg daily. In some embodiments, the effective amount of CBD is initiated at about 50 mg daily and titrated up to about 500 mg daily. The effective amount of CBD can be initiated at about 50 mg daily and titrated up to about 250 mg daily. In some embodiments, the effective amount of CBD is initiated at 250 mg daily. The effective amount of CBD can be initiated at 500 mg daily. In some embodiments, the 500 mg daily dose is administered to patients that weigh greater than 35 kg. The CBD can be administered in a single daily dose or in two daily doses. In some embodiments, the effective amount of CBD can be 390 mg in divided daily doses. The CBD can be formulated as a gel or an oil. In some embodiments, the CBD is formulated as a permeation-enhanced gel. The gel can contain between 1% (wt/wt) CBD to 7.5% (wt/wt) CBD. In some embodiments, the gel contains 4.2% (wt/wt) CBD. In some embodiments, the gel contains 7.5% (wt/wt) CBD. In some embodiments, the transdermal preparation can be a cream, a salve or an ointment. The CBD can be delivered by a bandage, pad or patch. In reference to the invention, “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenedi- of as well as to pharmaceutically acceptable salts, solvates, metabolites (e.g., cutaneous metabolites), and metabolic precursors thereof. The synthesis of CBD is described, for example, in Petilka et al., Hely. Chim. Acta, 52:1102 (1969) and in Mechoulam et al., J. Am. Chem. Soc., 87:3273 (1965), which are hereby incorporated by reference. As used herein, the term “transdermally administering” refers to contacting the CBD with the patient's or subject's skin under conditions effective for the CBD to penetrate the skin. The CBD can be in a gel form and can be pharmaceutically-produced as a clear, permeation-enhanced gel that is designed to provide controlled drug delivery transdermally with once- or twice-daily dosing. The CBD gel can between 1% (wt/wt) CBD to 7.5% (wt/wt) CBD. The CBD gel can have, for example, 4.2% (wt/wt) CBD or 7.5% (wt/wt) CBD). The CBD gel can be applied topically by the patient or caregiver to the patient's upper arm and shoulder, back, thigh, or any combination thereof. The CBD gel can include diluents and carriers as well as other conventional excipients, such as wetting agents, preservatives, and suspending and dispersing agents.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Carrier” or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regiment comprises a plurality of doses and at least two different time periods separating individual doses. In some embodiments, the therapeutic agent is administered continuously over a predetermined period. In some embodiments, the therapeutic agent is administered once a day (QD) or twice a day (BID).

By “does not detectably express” means that expression of a protein or gene cannot be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). Thus, a cell that “does not detectably express” a marker appears similar to the negative control for that marker. For gene expression, a gene “does not detectably express” if the presence of its mRNA cannot be visually detected on a standard agarose gel following standard PCR protocols.

Conversely, a cell “expresses” the protein or gene if it can be detected by the same method.

The term “culture expanded population” means a population of cells whose numbers have been increased by cell division in vitro. This term may apply to stem cell populations and non-stem cell populations alike.

The term “passaging” refers to the process of transferring a portion of cells from one culture vessel into a new culture vessel.

The term “cryopreserve” refers to preserving cells for long term storage in a cryoprotectant at low temperature.

The term “master cell bank” refers to a collection of cryopreserved cells. Such a cell bank may comprise stem cells, non-stem cells, and/or a mixture of stem cells and non-stem cells.

The invention teaches mans of “programming” the immune system to suppress autoimmunity through stimulation of T regulatory cells using cannabidiol as a means of activating T regulatory cells, and/or inducing proliferation, and/or inducing their de novo generation.

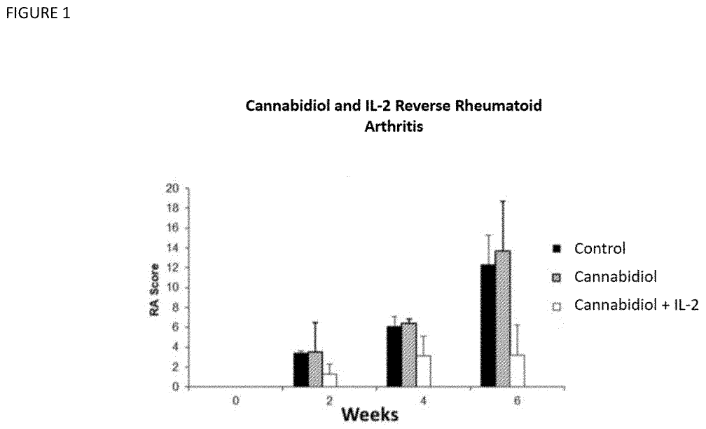

In one aspect, the disclosure addresses differentiation of naive T cells into stable regulatory T-cells (Tregs), using administration of cannabidiol, and/or combinations of cannabidiol with other immune regulatory agents such as low dose interleukin-2. This disclosure is based, in part, on the observation that administration of cannabidiol possesses ability to modulate T regulatory cell numbers in healthy animals and in animals suffering from autoimmunity. Accordingly, the inventors investigated the use of cannabidiol to stimulate T regulatory cells, as well as induce augmented immune suppressive activity of T regulatory cells. The results, described in more detail below, disrupt general paradigms that cannabidiol acts as a direct anti-inflammatory agent, but instead induces a state of “active immune tolerance”. Indeed it is reasonable to believe that the current discovery is markedly different than previous finings based on the fact that T regulatory cells can induce a state of “infectious tolerance” in which the tolerogenic process maintains itself after cessation of administration of the therapeutic agent. Such maintenance of a tolerogenic state has been previously described by numerous investigators [22-25]. The utilization of cannabidiol's newly discovered property of stimulating T regulatory cells is applicable to a wide variety of diseases. As the utility of this approach has become clear, one likely advantage to such therapeutic compounds is the enhanced likelihood for tolerance (e.g., reduced toxicity and side-effects) in the human (or other) subject considering that cannabidiol has been utilized for other medical means without knowledge of its ability to modulate T regulatory cells from naïve T cells. As used herein, the term “naive T cells” refers to lymphocytes that are typically derived from the thymus and express T cell receptors. The naive T cells have typically undergone the basic development in the bone marrow and further undergone the positive and negative processes of selection in the thymus. However, naive T cells have not encountered their cognate antigens yet in the periphery. The terms “activating” and/or “differentiation” refers to the process in which the naive T cell are caused to further develop into one of at least four distinct lineages of T cells characterized by distinct expression profiles and functions in vivo. See, e.g., FIG. 1. The term “activated” and/or “differentiated” can refer to the cell that had previously been naive but now has had an induction of specific gene expression such that it is identifiable as a particular activated/differentiated lineage. The terms can also refer to cells produced from the expansion of a T cell into a multitude of progeny cells by cell-division and which retain the identifiable markers for the particular activated/differentiated lineage. Thus, “activating a naive T cell” can refer to the production of an expanded cell population of differentiated T cells from the initial naive T cell, as well as the initial T cell after gene transcription has been induced.

For any embodiment, it can be readily determined the minimal amount of cannabidiol required to effect activation of the naive T cell into the desired differentiated T cell.

In one embodiment, cannabidiol is used to treat the naive T cell is differentiated into a cell with increased expression of FoxP3 compared to the naive T cell. The term “FoxP3” refers to a transcription factor also referred to as “forkhead box P3” or “scurfin”. While the precise control mechanism has not yet been established, FoxP3 protein belongs to the forkhead/winged-helix family of transcriptional regulators. In regulatory T cell model systems, FoxP3 occupies the promoters for genes involved in regulatory T-cell function, and may repress transcription of key genes following stimulation of T cell receptors. Accordingly, FoxP3 is known as a master regulator in the development of regulatory T cells (Tregs), which are involved in tolerance of antigens in the periphery and generally promote a protection against an inflammatory response. Examples of the FoxP3 protein include human (Entrez #:50943; RefSeq (mRNA): NM_001114377; RefSeq (amino acid): NP_001107849) and mouse (Entrez #: 20371; RefSeq (mRNA): NM_001199347; RefSeq (amino acid): NP_001186276). Many other FoxP3 protein and gene homologs are known for vertebrate animals, and their expression can be readily determined. As used herein, the term “increased” refers to a level of expression of the FoxP3 transcription factor that is detectably greater than that in a naive T cell, such as the initial naive T cell that is being differentiated, or other naive T cell obtained from the same individual (or an individual of the same species) as that as the initial naive T cell. Increased expression can be determined in terms of transcription of the underlying foxp3 gene or levels of functional FoxP3, using routine and established methods known in the art.

In one embodiment, the naive T cell is differentiated into a T regulatory cell (Treg). The term “Treg” refers to a lineage of T cells that promote or maintain tolerance to antigens, typically to self-antigens. Tregs have been previously referred to as “suppressor T cells.” Tregs generally suppress or downregulate induction and proliferation of effector T cells. As indicated above, Treg cells are typically characterized by the positive or increased expression of FoxP3. Tregs are also characterized by the additional positive or increased expression of CD4 and CD25. Thus, in one embodiment, the Treg is characterized by a state of CD4+, CD25+ and FoxP3+ expression.

In other embodiments, the contacting of the naive T cell results in an inhibition of a “Th17” inflammation phenotype by the differentiated T cells. For instance, the contacting of the naive T cell results in an inhibition or decrease in the expression of ROR.gamma.T, which is a marker for the Th17 (pro-inflammatory) phenotype of activated T cell normally involved in mucosal immunity In one embodiment herein, the naive T cell can be contacted in vitro in a culture medium. Typically, the culture medium contains factors commonly known to support and maintain T cell viability. The medium can also contain additional ingredients that are also known to promote T cell activation toward the desired differentiated lineage. Such additional ingredients are often referred to as “skewing” ingredients. Skewing factors can also include other microbiota metabolites (such as short-chain fatty acids, bile acids, polysaccharide A), dietary derived compounds (such as n3 polyunsaturated fatty acids, retinoic acid, and other vitamin-derivatives (VitD, VitC, etc.), polyphenols, quercetin, resveratrol, NSAIDS, TGF-.beta., IL-10, rapamycin, and IL-2. Other skewing factors that are useful for this purpose include curcumin, metformin/AMPK activators, PI3-kinase/Akt inhibitors, and PPAR agonists, as are known in the art. The invention teaches that cannabidiol augments ability of “skewing factors” to generate enhanced numbers of T regulatory cells.

In another aspect, the present disclosure provides a method of producing a Treg cell. In one embodiment, the method comprises contacting a naive T cell in vitro with a cannabidiol, wherein said cannabidiol can be contacted with the naive T cell as a component (e.g., additive) of a standard culture medium, as described above. The method can comprise the further culture and/or expansion of the activated T cell in its differentiated Treg state.

As described below, the inventors have demonstrated that the Tregs that are induced in vitro (“iTregs”) using the disclosed cannabidiol possess new features over induced Tregs (“iTregs”) produced using existing techniques. For example, the inventors have demonstrated that the iTregs resulting from the application of the TDMMs, such as indole, resulted in a stable iTreg that did not revert to a Th17 phenotype even in a “pro-inflammation” environment. Thus, in another aspect, the disclosure provides an induced T regulatory cell (iTreg). The iTreg is produced by the methods described herein. In some embodiments, the iTreg is produced by

contacting a naive T cell with cannabidiol. The iTreg can be the initial T cell after activation has occurred or a progeny cell in the differentiated state after expansion has occurred through one or more rounds of cell division from the initial T cell. In some embodiments, the iTreg exhibits increased stability in the Treg lineage as compared to iTregs that are induced using conventional means. For example, IL-4, IL-6, and IL-23 are all known to reduce typical Treg stability. This obstacle is overcome by iTregs. Accordingly, the iTreg lineage is less susceptible to induced instability by IL-4, IL-6, and IL-23. In some embodiments, the iTregs are distinguished from typical Tregs by a relative increased expression of CTLA4, CD62L, CD25, higher Foxp3, alpha4beta7, and/or CCR9, which can readily be determined by routine testing.

In another aspect, the present disclosure provides a method of increasing the stability of Treg cells by administration of cannabidiol. This refers to the lowered susceptibility of the Tregs to alter the Treg specific expression profiles in the context of pro-inflammatory cytokines and signaling, such as IL-4, IL-6, and IL-23, and the like. The Treg cells can be induced Tregs (iTregs) such as produced by the novel methods described herein or by existing methods in the art. Alternatively, the Tregs can be naturally occurring Tregs (nTregs). The term “nTregs” refers to the Tregs existing in vivo without prior in vitro intervention or transfer and are typically obtained from the thymus in humans. This method can be carried out in vitro by isolating and the Treg population, or alternatively expanding an iTreg population already ex vivo, and exposing the Tregs to the cannabidiol, or a precursor, prodrug, or acceptable salt thereof, as described herein. In some cases, if the target population is an iTreg population produced by the novel methods described herein, the iTregs will have already been exposed to the cannabidiol, or a precursor, prodrug, or acceptable salt thereof, and may or may not have additional exposure.

In another aspect, the present disclosure provides a method of reducing, preventing, ameliorating, attenuating, and/or otherwise treating inflammation in a subject in need thereof. General methods of using isolated or ex vivo/in vitro-differentiated Treg cells as part of adoptive T cell therapy to address inflammatory-related diseases are known. The method of the present aspect comprises administering to the subject the iTreg described immediately above, i.e., which is produced by contacting a naive T cell with a cannabidiol. In some embodiments, the subject suffers from or is susceptible to excessive or deleterious inflammation. In some embodiments, the subject has or is susceptible to allergies, inflammatory bowel disease, colitis, NSAID-enteropathy/ulceration, psoriasis, rheumatism, graft-versus-host disease, lupus, multiple sclerosis, and the like. In some embodiments, the subject has or is susceptible to a disease characterized by the role of mTor, stat3, akt, erk, jnk, stats, and/or smad2/3, which are targets of indole. Additionally or alternatively, the subject may suffer from deleterious inflammation due to a cancer or infection from a microbial or parasitic pathogen. The iTreg can be formulated for administration through any appropriate route according to known standards and methods. For example, the iTregs can be formulated for intra-peritoneal (IP), intravenous (IV), topical, parenteral, intradermal, transdermal, oral (e.g., via liquid or pill), inhaled (e.g., intranasal mist), and other appropriate routes of administration. In some embodiments, administration is directly to a mucosal region of the subject, such as in the digestive tract.

In some embodiments, the method comprises inducing the development of Tregs in vivo as described herein. In such embodiments, the subject can be administered an effective amount of cannabidiol, or a precursor, prodrug, or acceptable salt thereof. Administration of the cannabidiol can be in any appropriate route of administration. For example, the cannabidiol can be administered by intra-peritoneal (IP), intravenous (IV), topical, parenteral, intradermal, transdermal, oral (e.g., via liquid or pill), rectal, or respiratory (e.g., intranasal mist) routes. In preferred embodiments, the cannabidiol is ingested, e.g., via liquid or pill, etc. to facilitate delivery of the cannabidiol to the intestinal tract .

In some embodiments of the invention, the cannabinoid, such as cannabidiol or a prodrug of cannabidiol, is delivered systemically to achieve therapeutically effective plasma concentrations in a patient. However, cannabinoid oral dosage forms, including those comprising cannabidiol, must overcome several obstacles in order to achieve a therapeutically-effective systemic concentration. First, cannabinoids are generally highly lipophilic. Their limited water solubility thereby restricts the amount of cannabinoid available for absorption in the gastrointestinal tract. Second, cannabidiol, as with the other cannabinoids, undergoes substantial first-pass metabolism when absorbed from the human gastrointestinal tract. Finally, the oral bioavailability of any product is further diminished when a patient suffers from nausea or emesis, as either the patient avoids taking his oral medications or the oral dosage form does not remain in the gastrointestinal tract for a sufficient period of time to release the entire dose and achieve a therapeutic concentration. Therefore, in view of the foregoing, it would be desirable to systemically deliver therapeutically effective amounts of a cannabinoid, such as cannabidiol or cannabidiol prodrug, to a mammal in need thereof for the treatment of one or more medical conditions responsive to cannabinoids, including pancreatic cancer, pancreatitis, pain, nausea or appetite stimulation, by a route of administration that does not depend upon absorption from the gastrointestinal tract of the mammal. One non-oral route of administration for the systemic delivery of cannabidiol is transdermal administration. In addition, the epidermis and dermis of many mammals, such as humans and guinea pigs, contains enzymes which are capable of metabolizing active pharmaceutical agents which pass through the stratum corneum. The metabolic process occurring in the skin of mammals, such as humans, can be utilized to deliver pharmaceutically effective quantities of a cannabinoid, such as cannabidiol, to the systemic circulation of a mammal in need thereof. Described herein are prodrugs of cannabinoids, such as cannabidiol prodrugs, and compositions comprising prodrugs of cannabinoids that can be transdermally administered to a mammal, such as a human, so that the metabolic product resulting from metabolism in the skin is the cannabinoid which is systemically available for the treatment of a medical condition responsive to cannabinoid, for example pancreatic diseases, such as pancreatitis and pancreatic cancer. Unfortunately, due to its highly lipophilic nature, cannabidiol is poorly absorbed through membranes such as the skin of mammals, including humans. Therefore, the success of transdermally administering therapeutically effective quantities of cannabidiol to a mammal in need thereof within a reasonable time frame and over a suitable surface area has been substantially limited.

The use of cannabidiol for suppression of inflammation has previously been described in the art. The invention provides means of utilizing the anti-inflammatory effects of cannabidiol for stimulation of Treg cells. Once Treg cells are generated, the invention teaches that such Treg cells may be expanded. The utilization of cannabidiol for prevention of inflammation has been previously shown, for example, in one study, the effect of delta9 tetrahydrocannabinol (THC) and cannabidiol (CBD) on cytokine production in vitro by human leukemic T, B, eosinophilic and CD8+ NK cell lines as models was investigated. It was shown that THC decreased constitutive production of IL-8, MIP-lalpha, MIP-lbeta, and RANTES and phorbol ester stimulated production of TNF-alpha, GM-CSF and IFN-gamma by NK cells. It inhibited MIP-lbeta in HTLV-1 positive B-cells but tripled IL-8, MIP-1alpha and MIP-1beta in B-cells and MIP-lbeta in eosinophilic cells but doubled IL-8. Both cannabinoids strongly inhibited IL-10 production by HUT-78 T-cells. This suggests that cannabidiol may, in part, inhibit inflammation by suppressing chemotaxis [26]. This paper is interesting, because it is, in some ways, counterintuitive in that it is known that IL-10 is an anti-inflammatory cytokine which suppresses T cells , inhibits dendritic cell maturation and downregulates activity of NK cells. So if cannabidiol inhibits IL-10, it should actually be stimulating inflammation, not suppressing it.

The invention provides means of utilizing cannabidiol to prevent unwanted immune responses. For example, in pregnancy, “tolerogenic antigen presentation” occurs only through the indirect pathway of antigen presentation [27]. Other pathways of selective tolerogenesis in pregnancy include the stimulation of Treg cells, which have been demonstrated essential for successful pregnancy [28]. The invention, in one embodiment, teaches the modification of fibroblasts by transfection with MHC or MHC—like molecules in order to create an antigen presenting cell from said fibroblasts, wherein said antigen presenting cell is capable of inducing antigen-specific tolerance when administered into a host at a therapeutically sufficient concentration and frequency. In the context of cancer, depletion of tumor specific T cells, while sparing of T cells with specificities to other antigens has been demonstrated by the tumor itself or tumor associated cells [29-32]. This is the mechanism why which cancer can selectively induce a “hole in the repertoire” while allowing the host to be generally immunocompetent. Additionally, Treg cells have been demonstrated to actively suppress anti-tumor T cells, perhaps as a “back up” mechanism of tumor immune evasion [33-35]. At a clinical level the ability of tumors to inhibit peripheral T cell activity has been associated in numerous studies with poor prognosis [36-38]. Accordingly, in one embodiment of the invention the utilization of molecules that stimulate generation of Treg, as well as administration of molecules that expand Tregs which have been generated, are utilized. In one embodiment, fibroblasts are transfected with autoantigen together with interleukin-2 in order to enhance Treg generation. In other embodiments, interleukin 2 is administered systemically in order to enhance in vivo proliferation of Tregs.

Another natural example of tolerance that is utilized by the invention as a template for us of cannabidiol induced T regulatory cells is oral tolerance. Oral tolerance is the process by which ingested antigens induce generation of antigen-specific TGF-beta producing cells (called “Th3” by some) [39-41], as well as Treg cells [42, 43]. Ingestion of antigen, including the autoantigen collagen II [44], has been shown to induce inhibition of both T and B cell responses in a specific manner [45, 46]. It appears that induction of regulatory cells, as well as deletion/anergy of effector cells is associated with antigen presentation in a tolerogenic manner

. Remission of disease in animal models of RA [48], multiple sclerosis [49], and type I diabetes [50], has been reported by oral administration of autoantigens. Furthermore, clinical trials have shown signals of efficacy of oral tolerance in autoimmune diseases such as rheumatoid arthritis [51], autoimmune uveitis [52], and multiple sclerosis [53]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction. In some embodiments, oral tolerance is utilized together with the autoantigen transfected fibroblasts of the invention. For example, if a patient with type 1 diabetes is treated, said patient is administered cannabidiol, as well as cells that have been transfected with a diabetes specific autoantigen such as GAD65, additionally said cells may be transfected with tolerogenic molecules such as IL-10, and when said cell are administered, orally delivered GAD65 may be utilized in order to enhance the tolerogenic processes. In another embodiment, the invention teaches the transfection of cell with autoantigens combined with molecules associated with oral tolerogenesis such as TGF-beta.

In some embodiments, cells are transfected with biologically effective molecules in order to resemble the immune modulatory activities of mesenchymal stem cells. For example, it is known that these cells suppress T cell activation through inhibition of IL-2 receptor alpha (CD25) [54]. Accordingly in one embodiments, cells are transfected with one or more autoantigens as well as IL-2 receptors in order to “suck up” IL-2 so as to prevent T cells from being activated. In other embodiments fibroblasts are cultured under conditions used for culture of mesenchymal stem cells in order to endow said fibroblast with the properties of induction of division arrest [55, 56], induction of T cell anergy directly [57] or via immature DC [58], stimulation of apoptosis of activated T cells [59, 60], blockade of IL-2 signaling and induction of PGE2 production [61-66], induction of TGF-beta[67], production of HLA-G [68], expression of serine protease inhibitor 6 [69], stimulation of nitric oxide release [70-72], stimulation of indolamine 2,3 deoxygenase [73-76], expression of adenosine generating ectoenzymes such as CD39 and CD73 [77-79], Galectin expression[80, 81], induction of hemoxygenase 1[82, 83], activation of the PD1 pathway [80, 84-86], Fas ligand expression [87, 88], CD200 expression

, Th2 deviation [90-92], inhibition of Th17 differentiation [93-97], TSG-6 expression [98], NOTCH-1 expression [99], stimulation of Treg cell generation [100-107]. Mechanisms of Treg generation may be direct, or may be through modulation of DC. It has been reported by us and others, that activation of T cells in the absence of costimulatory signals leads to generation of immune suppressive CD4+ CD25+ T regulatory (Treg) cells [23, 108]. Thus local activation of immunity in lymph nodes would theoretically be associated with reduced costimulatory molecule expression DC after MSC administration, which may predispose to Treg generation. Conversely, it is known that Tregs are involved in maintaining DC in the DC2 phenotype [109]. Indeed numerous studies have demonstrated the ability of MSC to induce Treg cells [102, 103, 105, 110].

### EXAMPLES

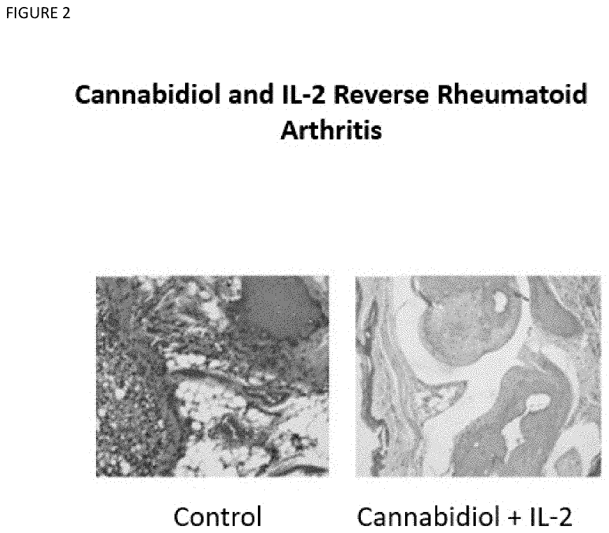

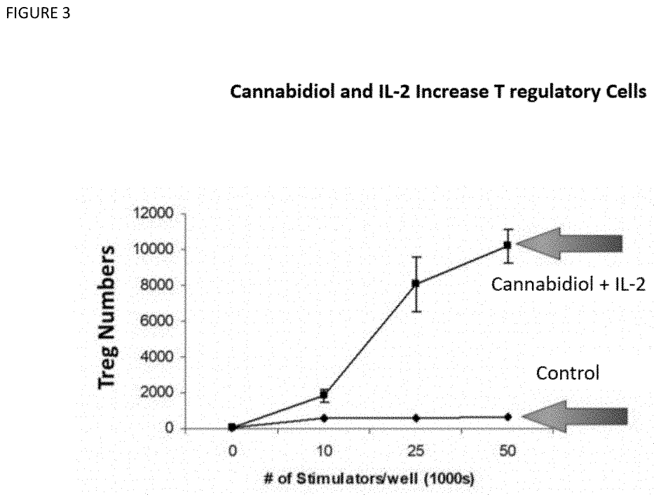

Collagen induced arthritis model was utilized and mice were administered cannabidiol (1 ug/mouse once per day) and IL-2 (2 units per mouse once a day). Reduction in arthritic activity, and joint inflammation was observed with the cannabidiol and IL-2 combination. Furthermore, an increase in FoxP3 positive cells was observed with the combination but not control. Results are shown in FIGS. 1, 2, and 3.

### REFERENCES

1. Ramsdell, F. and B. J. Fowlkes, Clonal deletion versus clonal anergy: the role of the thymus in inducing self tolerance. Science, 1990. 248(4961): p. 1342-8.
2. Apostolou, I., et al., Origin of regulatory T cells with known specificity for antigen. Nat Immunol, 2002. 3(8): p. 756-63.
3. Aschenbrenner, K., et al., Selection of Foxp3+ regulatory T cells specific for self antigen expressed and presented by Aire+medullary thymic epithelial cells. Nat Immunol, 2007. 8(4): p. 351-8.
4. Aluvihare, V. R., M. Kallikourdis, and A. G. Betz, Regulatory T cells mediate maternal tolerance to the fetus. Nat Immunol, 2004. 5(3): p. 266-71.
5. Somerset, D. A., et al., Normal human pregnancy is associated with an elevation in the immune suppressive CD25+ CD4+ regulatory T-cell subset. Immunology, 2004. 112(1): p. 38-43.
6. Campos-Mora, M., et al., CD4+ Foxp3+ T Regulatory Cells Promote Transplantation Tolerance by Modulating Effector CD4+ T Cells in a Neuropilin-1-Dependent Manner. Front Immunol, 2019. 10: p. 882.
7. Lalfer, M., et al., Foxp3(+) Regulatory and Conventional CD4(+) T Cells Display Similarly High Frequencies of Alloantigen-Reactive Cells. Front Immunol, 2019. 10: p. 521.
8. Wang, K., et al., Different phenotypes of CD4(+)CD25(+)Foxp3(+) regulatory T cells in recipients post liver transplantation. Int Immunopharmacol, 2019. 69: p. 194-201.
9. Herrera-Gomez, F., et al., The number of FoxP3 regulatory T cells in the circulation may be a predictive biomarker for kidney transplant recipients: A multistage systematic review. Int Immunopharmacol, 2018. 65: p. 483-492.
10. Leung, C. S., et al., Single-cell transcriptomics reveal that PD-1 mediates immune tolerance by regulating proliferation of regulatory T cells. Genome Med, 2018. 10(1): p. 71.
11. Zhu, X., et al., CD4+CD25+Tregs express an increased LAG-3 and CTLA-4 in anterior chamber-associated immune deviation. Graefes Arch Clin Exp Ophthalmol, 2007. 245(10): p. 1549-57.
12. Zhang, H., et al., Involvement of Foxp3-expressing CD4+ CD25+ regulatory T cells in the development of tolerance induced by transforming growth factor-beta2-treated antigen presenting cells. Immunology, 2008. 124(3): p. 304-14.
13. He, H., et al., Upregulation of CD94 on CD8+T cells in anterior chamber-associated immune deviation. BMC Immunol, 2008. 9: p. 53.
14. Saban, D. R., et al., The role of ACAID and CD4+CD25+FOXP3+ regulatory T cells on CTL function against MHC alloantigens. Mol Vis, 2008. 14: p. 2435-42.
15. Hori, J., et al., GITR ligand-mediated local expansion of regulatory T cells and immune privilege of corneal allografts. Invest Ophthalmol Vis Sci, 2010. 51(12): p. 6556-65.
16. Ji, S. X., X. L. Yin, and P. Z. Yang, Effect of CD4(+)CD25(+) regulatory T cells in the development of anterior chamber-associated immune deviation. Int J Ophthalmol, 2011. 4(1): p. 19-25.
17. Zhang, Y., et al., Effects of human umbilical cord-derived mesenchymal stem cells on anterior chamber-associated immune deviation. Int Immunopharmacol, 2013. 15(1): p. 114-20.
18. Yan, F., et al., Tolerogenic dendritic cells suppress murine corneal allograft rejection by modulating CD28/CTLA-4 expression on regulatory T cells. Cell Biol Int, 2014. 38(7): p. 835-48.
19. Wang, T., et al., c-Rel is Required for the Induction of pTregs in the Eye but Not in the Gut Mucosa. Immunol Invest, 2016. 45(8): p. 776-786.
20. Kunishige, T., et al., Protective Role of ICOS and ICOS Ligand in Corneal Transplantation and in Maintenance of Immune Privilege. Invest Ophthalmol Vis Sci, 2016. 57(15): p. 6815-6823.
21. Zenclussen, A. C., et al., Abnormal T-cell reactivity against paternal antigens in spontaneous abortion: adoptive transfer of pregnancy-induced CD4+CD25+ T regulatory cells prevents fetal rejection in a murine abortion model. Am J Pathol, 2005. 166(3): p. 811-22.
22. Min, W. P., et al., Inhibitory feedback loop between tolerogenic dendritic cells and regulatory T cells in transplant tolerance. J Immunol, 2003. 170(3): p. 1304-12.
23. Zhang, X., et al., Generation of therapeutic dendritic cells and regulatory T cells for preventing allogeneic cardiac graft rejection. Clin Immunol, 2008. 127(3): p. 313-21.
24. Zhou, X., et al., Tolerogenic Vaccine Composited with Islet Derived Multipeptides and Cyclosporin A Induces pTreg and Prevents Type 1 Diabetes in Murine Model. Hum Vaccin Immunother, 2019.
25. Tabansky, I., et al., Targeting DEC-205(−)DCIR2(+) dendritic cells promotes immunological tolerance in proteolipid protein-induced experimental autoimmune encephalomyelitis. Mol Med, 2018. 24(1): p. 17.
26. Srivastava, M. D., B. I. Srivastava, and B. Brouhard, Delta9 tetrahydrocannabinol and cannabidiol alter cytokine production by human immune cells. Immunopharmacology, 1998. 40(3): p. 179-85.
27. Erlebacher, A., et al., Constraints in antigen presentation severely restrict T cell recognition of the allogeneic fetus. J Clin Invest, 2007. 117(5): p. 1399-411.
28. Chen, T., et al., Self-specific memory regulatory T cells protect embryos at implantation in mice. J Immunol, 2013. 191(5): p. 2273-81.
29. Harimoto, H., et al., Inactivation of tumor-specific CD8(+) CTLs by tumor-infiltrating tolerogenic dendritic cells. Immunol Cell Biol, 2013. 91(9): p. 545-55.
30. Ney, J. T., et al., Autochthonous liver tumors induce systemic T cell tolerance associated with T cell receptor down-modulation. Hepatology, 2009. 49(2): p. 471-81.
31. Cheung, A. F., et al., Regulated expression of a tumor-associated antigen reveals multiple levels of T-cell tolerance in a mouse model of lung cancer. Cancer Res, 2008. 68(22): p. 9459-68.
32. Bal, A., et al., Rapid tolerization of virus-activated tumor-specific CD8+ T cells in prostate tumors of TRAMP mice. Proc Natl Acad Sci U S A, 2008. 105(35): p. 13003-8.
33. Jacobs, J. F., et al., Regulatory T cells in melanoma: the final hurdle towards effective immunotherapy? Lancet Oncol, 2012. 13(1): p. e32-42.
34. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
35. Donkor, M. K., et al., T cell surveillance of oncogene-induced prostate cancer is impeded by T cell-derived TGF-beta1 cytokine. Immunity, 2011. 35(1): p. 123-34.
36. Whiteside, T. L., Down-regulation of zeta-chain expression in T cells: a biomarker of prognosis in cancer? Cancer Immunol Immunother, 2004. 53(10): p. 865-78.
37. Whiteside, T. L., Signaling defects in T lymphocytes of patients with malignancy. Cancer Immunol Immunother, 1999. 48(7): p. 346-52.
38. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
39. Faria, A. M. and H. L. Weiner, Oral tolerance. Immunol Rev, 2005. 206: p. 232-59.
40. Weiner, H. L., Induction and mechanism of action of transforming growth factor-beta-secreting Th3 regulatory cells. Immunol Rev, 2001. 182: p. 207-14.
41. Fukaura, H., et al., Induction of circulating myelin basic protein and proteolipid protein-specific transforming growth factor-betal -secreting Th3 T cells by oral administration of myelin in multiple sclerosis patients. J Clin Invest, 1996. 98(1): p. 70-7.
42. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.
43. Yamashita, H., et al., Overcoming food allergy through acquired tolerance conferred by transfer of Tregs in a murine model. Allergy, 2012. 67(2): p. 201-9.
44. Park, K. S., et al., Type II collagen oral tolerance; mechanism and role in collagen-induced arthritis and rheumatoid arthritis. Mod Rheumatol, 2009.
45. Womer, K. L., et al., A pilot study on the immunological effects of oral administration of donor major histocompatibility complex class II peptides in renal transplant recipients. Clin Transplant, 2008. 22(6): p. 754-9.
46. Faria, A. M. and H. L. Weiner, Oral tolerance: therapeutic implications for autoimmune diseases. Clin Dev Immunol, 2006. 13(2-4): p. 143-57.
47. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.
48. Thompson, H. S., et al., Suppression of collagen induced arthritis by oral administration of type II collagen: changes in immune and arthritic responses mediated by active peripheral suppression. Autoimmunity, 1993. 16(3): p. 189-99.
49. Song, F., et al., The thymus plays a role in oral tolerance induction in experimental autoimmune encephalomyelitis. Ann N Y Acad Sci, 2004. 1029: p. 402-4.
50. Hanninen, A. and L. C. Harrison, Mucosal tolerance to prevent type 1 diabetes: can the outcome be improved in humans? Rev Diabet Stud, 2004. 1(3): p. 113-21.
51. Wei, W., et al., A multicenter, double-blind, randomized, controlled phase III clinical trial of chicken type II collagen in rheumatoid arthritis. Arthritis Res Ther, 2009. 11(6): p. R180.
52. Thurau, S. R., et al., Molecular mimicry as a therapeutic approach for an autoimmune disease: oral treatment of uveitis-patients with an MHC peptide crossreactive with autoantigen--first results. Immunol Lett, 1997. 57(1-3): p. 193-201.
53. Weiner, H. L., et al., Double-blind pilot trial of oral tolerization with myelin antigens in multiple sclerosis. Science, 1993. 259(5099): p. 1321-4.
54. Le Blanc, K., et al., Mesenchymal stem cells inhibit the expression of CD25 (interleukin-2 receptor) and CD38 on phytohaemagglutinin-activated lymphocytes. Scand J Immunol, 2004. 60(3): p. 307-15.
55. Glennie, S., et al., Bone marrow mesenchymal stem cells induce division arrest anergy of activated T cells. Blood, 2005. 105(7): p. 2821-7.
56. Kim, J. A., et al., The inhibition of T-cells proliferation by mouse mesenchymal stem cells through the induction of p16INK4A-cyclin D1/cdk4 and p21waf1, p27kip1-cyclin E/cdk2 pathways. Cell Immunol, 2007. 245(1): p. 16-23.
57. Zappia, E., et al., Mesenchymal stem cells ameliorate experimental autoimmune encephalomyelitis inducing T-cell anergy. Blood, 2005. 106(5): p. 1755-61.
58. Wang, Q., et al., Murine bone marrow mesenchymal stem cells cause mature dendritic cells to promote T-cell tolerance. Scand J Immunol, 2008. 68(6): p. 607-15.
59. Plumas, J., et al., Mesenchymal stem cells induce apoptosis of activated T cells. Leukemia, 2005. 19(9): p. 1597-604.
60. Lim, J. H., et al., Immunomodulation of delayed-type hypersensitivity responses by mesenchymal stem cells is associated with bystander T cell apoptosis in the draining lymph node. J Immunol, 2010. 185(7): p. 4022-9.
61. Rasmusson, I., et al., Mesenchymal stem cells inhibit lymphocyte proliferation by mitogens and alloantigens by different mechanisms. Exp Cell Res, 2005. 305(1): p. 33-41.
62. Xu, G., et al., Immunosuppressive properties of cloned bone marrow mesenchymal stem cells. Cell Res, 2007. 17(3): p. 240-8.
63. English, K., et al., Cell contact, prostaglandin E(2) and transforming growth factor beta 1 play non-redundant roles in human mesenchymal stem cell induction of CD4+CD25(High) forkhead box P3+regulatory T cells. Clin Exp Immunol, 2009. 156(1): p. 149-60.
64. Spaggiari, G. M., et al., MSCs inhibit monocyte-derived DC maturation and function by selectively interfering with the generation of immature DCs: central role of MSC-derived prostaglandin E2. Blood, 2009. 113(26): p. 6576-83.
65. Yanez, R., et al., Prostaglandin E2 plays a key role in the immunosuppressive properties of adipose and bone marrow tissue-derived mesenchymal stromal cells. Exp Cell Res, 2010. 316(19): p. 3109-23.
66. Zafranskaya, M., et al., PGE2 Contributes to In vitro MSC-Mediated Inhibition of Non-Specific and Antigen-Specific T Cell Proliferation in MS Patients. Scand J Immunol, 2013. 78(5): p. 455-62.
67. Nasef, A., et al., Identification of IL-10 and TGF-beta transcripts involved in the inhibition of T-lymphocyte proliferation during cell contact with human mesenchymal stem cells. Gene Expr, 2007. 13(4-5): p. 217-26.
68. Magatti, M., et al., Human amnion mesenchyme harbors cells with allogeneic T-cell suppression and stimulation capabilities. Stem Cells, 2008. 26(1): p. 182-92.
69. El Haddad, N., et al., Mesenchymal stem cells express serine protease inhibitor to evade the host immune response. Blood, 2011. 117(4): p. 1176-83.
70. Sato, K., et al., Nitric oxide plays a critical role in suppression of T-cell proliferation by mesenchymal stem cells. Blood, 2007. 109(1): p. 228-34.
71. Oh, I., et al., Interferon-gamma and NF-kappaB mediate nitric oxide production by mesenchymal stromal cells. Biochem Biophys Res Commun, 2007. 355(4): p. 956-62.
72. Ren, G., et al., Mesenchymal stem cell-mediated immunosuppression occurs via concerted action of chemokines and nitric oxide. Cell Stem Cell, 2008. 2(2): p. 141-50.
73. DelaRosa, O., et al., Requirement of IFN-gamma-mediated indoleamine 2,3-dioxygenase expression in the modulation of lymphocyte proliferation by human adipose-derived stem cells. Tissue Eng Part A, 2009. 15(10): p. 2795-806.
74. Tipnis, S., C. Viswanathan, and A. S. Majumdar, Immunosuppressive properties of human umbilical cord-derived mesenchymal stem cells: role of B7-H1 and IDO. Immunol Cell Biol, 2010. 88(8): p. 795-806.
75. Ge, W., et al., Regulatory T-cell generation and kidney allograft tolerance induced by mesenchymal stem cells associated with indoleamine 2,3-dioxygenase expression. Transplantation, 2010. 90(12): p. 1312-20.
76. Francois, M., et al., Human MSC suppression correlates with cytokine induction of indoleamine 2,3-dioxygenase and bystander M2 macrophage differentiation. Mol Ther, 2012. 20(1): p. 187-95.
77. Sattler, C., et al., Inhibition of T-cell proliferation by murine multipotent mesenchymal stromal cells is mediated by CD39 expression and adenosine generation. Cell Transplant, 2011. 20(8): p. 1221-30.
78. Saldanha-Araujo, F., et al., Mesenchymal stromal cells up-regulate CD39 and increase adenosine production to suppress activated T-lymphocytes. Stem Cell Res, 2011. 7(1): p. 66-74.
79. Barry, F., et al., The SH-3 and SH-4 antibodies recognize distinct epitopes on CD73 from human mesenchymal stem cells. Biochem Biophys Res Commun, 2001. 289(2): p. 519-24.
80. Xue, Q., et al., The negative co-signaling molecule b7-h4 is expressed by human bone marrow-derived mesenchymal stem cells and mediates its T-cell modulatory activity. Stem Cells Dev, 2010. 19(1): p. 27-38.
81. Gieseke, F., et al., Human multipotent mesenchymal stromal cells use galectin-1 to inhibit immune effector cells. Blood, 2010. 116(19): p. 3770-9.
82. Chabannes, D., et al., A role for heme oxygenase-1 in the immunosuppressive effect of adult rat and human mesenchymal stem cells. Blood, 2007. 110(10): p. 3691-4.
83. Mougiakakos, D., et al., The impact of inflammatory licensing on heme oxygenase-1-mediated induction of regulatory T cells by human mesenchymal stem cells. Blood, 2011. 117(18): p. 4826-35.
84. Augello, A., et al., Bone marrow mesenchymal progenitor cells inhibit lymphocyte proliferation by activation of the programmed death 1 pathway. Eur J Immunol, 2005. 35(5): p. 1482-90.
85. Sheng, H., et al., A critical role of IFN gamma in priming MSC-mediated suppression of T cell proliferation through up-regulation of B7-H1. Cell Res, 2008. 18(8): p. 846-57.
86. Luz-Crawford, P., et al., Mesenchymal stem cells repress Th17 molecular program through the PD-1 pathway. PLoS One, 2012. 7(9): p. e45272.
87. Akiyama, K., et al., Mesenchymal-stem-cell-induced immunoregulation involves FAS-ligand-/FAS-mediated T cell apoptosis. Cell Stem Cell, 2012. 10(5): p. 544-55.
88. Gu, Y. Z., et al., Different roles of PD-L1 and FasL in immunomodulation mediated by human placenta-derived mesenchymal stem cells. Hum Immunol, 2013. 74(3): p. 267-76.
89. Najar, M., et al., Characterization and functionality of the CD200-CD200R system during mesenchymal stromal cell interactions with T-lymphocytes. Immunol Lett, 2012. 146(1-2): p. 50-6.
90. Batten, P., et al., Human mesenchymal stem cells induce T cell anergy and downregulate T cell allo-responses via the TH2 pathway: relevance to tissue engineering human heart valves. Tissue Eng, 2006. 12(8): p. 2263-73.
91. Lu, X., et al., Immunomodulatory effects of mesenchymal stem cells involved in favoring type 2 T cell subsets. Transpl Immunol, 2009. 22(1-2): p. 55-61.
92. Zanone, M. M., et al., Human mesenchymal stem cells modulate cellular immune response to islet antigen glutamic acid decarboxylase in type 1 diabetes. J Clin Endocrinol Metab, 2010. 95(8): p. 3788-97.
93. Ko, E., et al., Mesenchymal stem cells inhibit the differentiation of CD4+ T cells into interleukin-17-secreting T cells. Acta Haematol, 2008. 120(3): p. 165-7.
94. Rafei, M., et al., Mesenchymal stromal cells ameliorate experimental autoimmune encephalomyelitis by inhibiting CD4 Th17 T cells in a CC chemokine ligand 2-dependent manner. J Immunol, 2009. 182(10): p. 5994-6002.
95. Tatara, R., et al., Mesenchymal stromal cells inhibit Th17 but not regulatory T-cell differentiation. Cytotherapy, 2011. 13(6): p. 686-94.
96. Duffy, M. M., et al., Mesenchymal stem cell inhibition of T-helper 17 cell- differentiation is triggered by cell-cell contact and mediated by prostaglandin E2 via the EP4 receptor. Eur J Immunol, 2011. 41(10): p. 2840-51.
97. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
98. Kota, D. J., et al., TSG-6 produced by hMSCs delays the onset of autoimmune diabetes by suppressing Th1 development and enhancing tolerogenicity. Diabetes, 2013. 62(6): p. 2048-58.
99. Del Papa, B., et al., Notch1 modulates mesenchymal stem cells mediated regulatory T-cell induction. Eur J Immunol, 2013. 43(1): p. 182-7.
100. Maccario, R., et al., Interaction of human mesenchymal stem cells with cells involved in alloantigen-specific immune response favors the differentiation of CD4+ T-cell subsets expressing a regulatory/suppressive phenotype. Haematologica, 2005. 90(4): p. 516-25.
101. Prevosto, C., et al., Generation of CD4+ or CD8+ regulatory T cells upon mesenchymal stem cell-lymphocyte interaction. Haematologica, 2007. 92(7): p. 881-8.
102. Di Ianni, M., et al., Mesenchymal cells recruit and regulate T regulatory cells. Exp Hematol, 2008. 36(3): p. 309-18.
103. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
104. Boumaza, I., et al., Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun, 2009. 32(1): p. 33-42.
105. Ye, Z., et al., Immunosuppressive effects of rat mesenchymal stem cells: involvement of CD4+CD25+ regulatory T cells. Hepatobiliary Pancreat Dis Int, 2008. 7(6): p. 608-14.
106. Madec, A. M., et al., Mesenchymal stem cells protect NOD mice from diabetes by inducing regulatory T cells. Diabetologia, 2009. 52(7): p. 1391-9.
107. Melief, S. M., et al., Multipotent stromal cells induce human regulatory T cells through a novel pathway involving skewing of monocytes toward anti-inflammatory macrophages. Stem Cells, 2013. 31(9): p. 1980-91.
108. Ichim, T. E., R. Zhong, and W. P. Min, Prevention of allograft rejection by in vitro generated tolerogenic dendritic cells. Transpl Immunol, 2003. 11(3-4): p. 295-306.
109. Tiemessen, M. M., et al., CD4+CD25+Foxp3+ regulatory T cells induce alternative activation of human monocytes/macrophages. Proc Natl Acad Sci U S A, 2007. 104(49): p. 19446-51.
110. Gonzalez-Rey, E., et al., Human adipose-derived mesenchymal stem cells reduce inflammatory and T-cell responses and induce regulatory T cells in vitro in rheumatoid arthritis. Ann Rheum Dis, 2009.

1. A method of inducing T regulatory cells in vivo comprising the steps of: a) providing cannabidiol to a mammal at a concentration sufficient to induce expression of Foxp3 in T cells possessing the marker CD4; and b) altering the dose of cannabidiol as needed in order to maintain an general increase in cells expressing CD4 and FoxP3.

2. The method of claim 1, wherein said Treg cells are further expanded in vivo by administration of low dose interleukin-2.

3. The method of claim 1, wherein said Treg cells are further expanded in vivo by administration of antibody to CD45RB.

4. The method of claim 1, wherein said Treg cells are further expanded in vivo by administration of immature dendritic cells.

5. The method of claim 4, wherein said immature dendritic cells expression low levels CD40 as compared to steady state dendritic cells.

6. The method of claim 4, wherein said immature dendritic cells expression low levels CD80 as compared to steady state dendritic cells.

7. The method of claim 4, wherein said immature dendritic cells expression low levels CD86 as compared to steady state dendritic cells.

8. The method of claim 4, wherein said immature dendritic cells expression low levels IL-12 as compared to steady state dendritic cells.

9. The method of claim 4, wherein said immature dendritic cells expression higher levels of IL-10 as compared to steady state dendritic cells.

10. The method of claim 4, wherein said immature dendritic cells expression higher levels of PD-L1 as compared to steady state dendritic cells.

11. The method of claim 1, wherein said mesenchymal stem cells are administered together with cannabidiol.

12. The method of claim 11, wherein said mesenchymal stem cells are obtained from adipose tissue.

13. The method of claim 11, wherein said mesenchymal stem cells are obtained from bone marrow tissue.

14. The method of claim 11, wherein said mesenchymal stem cells are obtained from endometrial tissue.

15. The method of claim 11, wherein said mesenchymal stem cells are obtained from peripheral blood.

16. The method of claim 15, wherein said mobilization of mesenchymal stem cells is accomplished by administration of an agent selected from a group comprising of: a) G-CSF; b) GM-CSF; c) M-CSF; d) Mozibil; and e) FLT-3 ligand.

17. The method of claim 1, wherein said activation of Treg cells is accomplished in a mammal suffering from an inflammatory condition.

18. The method of claim 17, wherein said inflammatory condition is osteoarthritis.

19. The method of claim 17, wherein said inflammatory condition is rheumatoid arthritis.

20. The method of claim 17, wherein said inflammatory condition is an autoimmune disease.
